(19) United States
(12) Patent Application Publication
(10) Pub. No.: US 2014/0018353 A1
(43) Pub. Date: Jan. 16, 2014

(54) METHOD OF TREATING B-CELL MALIGNANT CANCERS AND T-CELL MALIGNANT CANCERS USING THIENOTRIAZOLODIAZEPINE COMPOUNDS

(71) Applicant: Oncoethix SA, Lousanne (CH)

(72) Inventors: Francesco Bertoni, Bellinzona (CH); Paola Bonetti, Milan (IT)

(21) Appl. No.: 13/926,029

(22) Filed: Jun. 25, 2013

Related U.S. Application Data
(60) Provisional application No. 61/663,885, filed on Jun. 25, 2012.
(60) Provisional application No. 61/670,918, filed on Jul. 12, 2012.

(51) Int. Cl. A61K 31/551

(52) U.S. Cl. CPC A61K 31/551
USPC 514/220

(57) ABSTRACT

A method of treating B-cell malignant cancers or T-cell malignant cancers in a mammal by administering to a patient a pharmaceutically acceptable amount of a composition comprising (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide. The B-cell malignant cancers include diffuse large B-cell lymphoma and splenic marginal zone lymphoma. The T-cell malignant cancers include anaplastic large T-cell lymphoma.

### CROSS REFERENCES

This application claims the benefit of U.S. Provisional Application Ser. No. 61/663,885, filed Jun. 25, 2012, and U.S. Provisional Application Ser. No. 61/670,918, filed Jul. 12, 2012, each of which are incorporated herein by reference in their entirety.

### FIELD OF THE INVENTION

The present invention relates to methods of treating B-cell malignant cancers and T-cell malignant cancers using pharmaceutically acceptable amounts of a composition comprising a thienotriazolodiazepine compound.

### BACKGROUND OF THE INVENTION

Bromodomain-containing proteins play an important role in gene expression regulation, via chromatin structure remodelling. Antitumor activity has been reported in acute and chronic hematological malignancies, including B-cell and T-cell malignancies, using inhibitors of BRD2/3/4, members of the Bromodomain and Extraterminal (BET) family. B-cell malignancies, which are also known as B-cell neoplasms or B-cell lymphomas, are cancers that occur when B-cells are overproduced or are malignant. B-cell malignancies include diffuse large B-cell lymphoma (DLBCL), mantle cell lymphoma (MCL), splenic marginal zone lymphoma (SMZL), and multiple myeloma (MM). T-cell malignancies, such as anaplastic large T-cell lymphoma, are a heterogeneous group of lymphoid neoplasms representing malignant transformation of the T lymphocytes. The present disclosure presents methods of treating certain B-cell malignant cancers and T-cell malignant cancers.

### SUMMARY OF THE INVENTION

In one embodiment, the present invention provides for a method of treating B-cell malignant cancers or T-cell malignant cancers in a mammal by administering to a patient a pharmaceutically acceptable amount of a composition comprising (S)-2-[4-(4-chlorophenyl)-2,3,9-trimethyl-6H-thieno[3,2-f][1,2,4]triazolo -[4,3-a][1,4]diazepin-6-yl]-N-(4-ydroxyphenyl)acetamide having the structure of Formula 2:

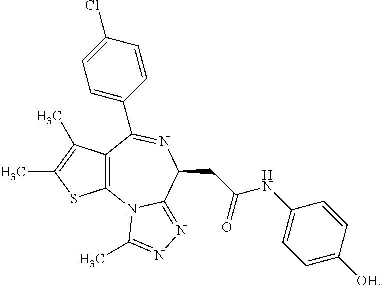 [SMILES: Cc1sc2c(c1C)C(c1ccc(Cl)cc1)=N[C@@H](CC(=O)Nc1ccc(O)cc1)c1nnc(C)n1-2]

In one embodiment, the present invention provides for a method of treating B-cell malignant cancers or T-cell malignant cancers in a patient by administering to a patient a pharmaceutically acceptable amount of a composition comprising a thienotriazolodiazepine compound represented by Formula 2 wherein the patient is a human.

In one embodiment, the present invention provides for a method of treating diffuse large B-cell lymphoma in a mammal by administering to a patient a pharmaceutically acceptable amount of a composition comprising a thienotriazolodiazepine compound represented by Formula 1.

In one embodiment, the present invention provides for a method of treating splenic marginal zone lymphoma in a mammal by administering to a patient a pharmaceutically acceptable amount of a composition comprising a thienotriazolodiazepine compound represented by Formula 1.

In one embodiment, the present invention provides for a method of treating anaplastic large T-cell lymphoma in a mammal by administering to a patient a pharmaceutically acceptable amount of a composition comprising a thienotriazolodiazepine compound represented by Formula 1.

### BRIEF DESCRIPTION OF THE DRAWINGS

The foregoing summary, as well as the following detailed description of the invention, will be better understood when read in conjunction with the appended drawings.

FIG. 1 illustrates the effect of Formula 2 on the proliferation of four MCL cell lines. Y-axis, percentage of viable cells. X-axis, doses of Formula 2 in μM.

FIG. 2A illustrates protein expression levels of BRD2, BRD3, and BRD4 in four MCL cell lines.

FIG. 2B illustrates RNA expression levels of BRD2, BRD3, and BRD4 in four MCL cell lines. X-axis, cell lines. Y-axis, mRNA quantities, relative to GAPDH.

FIG. 3A illustrates the effect of Formula 2 on the proliferation of DLBCL cell lines. Cells were exposed for 72 h to the indicated concentration of the drug. Viable cell number was determined by MTT assay. Y-axis, percentage of viable cells. X-axis, doses of Formula 2 in μM.

FIG. 3B illustrates the effect of Formula 2 on the proliferation of DLBCL cell lines (as in FIG. 3A, but expanded to show the lower doses). Cells were exposed for 72 h to the indicated concentration of the drug. Viable cell number was determined by MTT assay. Y-axis, percentage of viable cells. X-axis, doses of Formula 2 in μM.

FIG. 4A illustrates protein expression levels of BRD2, BRD3, and BRD4 in DLBCL cell lines.

FIG. 4B illustrates RNA expression levels of BRD2, BRD3, and BRD4 in DLBCL cell lines. X-axis, cell lines. Y-axis, mRNA quantities, relative to GAPDH.

FIG. 5A illustrates cell death in DLBCL cell lines exposed to Formula 2 for 24 hours. X-axis, cell lines. Y axis, percentage of PI-positive cells.

FIG. 5B illustrates cell death in DLBCL cell lines exposed to Formula 2 for 72 hours. X-axis, cell lines. Y axis, percentage of PI-positive cells.

FIG. 6 illustrates cell cycle alterations induced by Formula 2 in DLBCL cell lines. Representative histograms of flow cytometry profiles of untreated control cells and cells treated for 24 h with different doses of Formula 2. X-axis, cell lines. Y-axis, percentage of cells in each cell cycle phase.

FIG. 7 illustrates representative flow cytometry profiles of untreated control cells and SU-DHL-6 cells treated for 24 hours with 0.2 in μM of Formula 2.

FIGS. 8A-8F illustrate reductions of MYC, CAD, and NUC mRNA levels after increasing doses of Formula 2 in six DLBCL cell lines.

FIG. 9 illustrates the partial down-regulation of NFκB target genes after treatment with Formula 2 in two DLBCL cell lines.

FIG. 10 illustrates gene expression profiles before and after exposure to increasing concentrations of Formula 2 with increasing time in two sensitive models (SU-DHL2 and DoHH₂).

FIG. 11 illustrates the down-regulation of c-MYC in two of three DLBCL cell lines after 1 hour of treatment with Formula 2.

FIGS. 12A-12C illustrate the effect of Formula 2 on the proliferation of DLBCL cell lines, DoHH2, U-2932 and SU-DHL-6, with time after 24 hour treatment with IC50 dose of Formula 2 followed by wash-out.

FIG. 13 illustrates the effect on three DLBCL cell lines after six (6) days of exposure of Formula 2.

FIG. 14 illustrates the effect of Formula 2 on the proliferation of MM cell lines and BRD2, BRD3 and BRD4 expression. Y-axis, percentage of viable cells; X-axis, doses of Formula 2 in μM and protein levels of BRD2, BRD3 and BRD4 in MM cell lines.

FIG. 15 illustrates cell death in MM cell lines exposed to Formula 2 for 24 hours. X-axis, cell lines. Y axis, percentage of PI-positive cells.

FIG. 16 illustrates cell cycle alterations induced by Formula 2 in MM cell lines. Representative histograms of flow cytometry profiles of untreated control cells and cells treated for 24 h with different doses of Formula 2. X-axis, cell lines. Y-axis, percentage of cells in each cell cycle phase

FIG. 17 illustrates reduction of MYC mRNA after increasing doses of Formula 2 in two MM cell lines.

FIG. 18 illustrates that Formula 2 displays a cytostatic effect more than cytotoxic effect on MM cell lines exposed to Formula 2 for 24 hours. X-axis, cell lines. Y-axis, percentage of PI-positive cells.

FIG. 19 illustrates reductions of MYC mRNA levels after increasing doses of Formula 2 in two MM cell lines, MM1S and RPMI 8226.

FIGS. 20A and 20B illustrate cell cycle alterations induced by Formula 2 in MM cell lines. Representative histograms of flow cytometry profiles of untreated control cells and cells treated for 24 h with different doses of Formula 2. X-axis, cell lines. Y-axis, percentage of cells in each cell cycle phase.

FIG. 21 illustrates a plot of all IC50 values for each testing cell line sorted by increasing sensitivity to Formula 2.

FIG. 22 illustrates the effect of Formula 2 on the proliferation of SMZL cell lines. Y-axis, percentage of viable cells. X-axis, doses of Formula 2 in μM.

FIG. 23 illustrates the effect of Formula 2 on the proliferation of ALCL cell lines. Y-axis, percentage of viable cells. X-axis, doses of Formula 2 in μM.

FIG. 24A illustrates RNA expression levels of BRD2, BRD3, and BRD4 in various ALCL cell lines. X-axis, cell lines. Y-axis, mRNA quantities, relative to GAPDH.

FIG. 24B illustrates protein expression levels of BRD2, BRD3, and BRD4 in various ALCL cell lines.

FIGS. 25A-25E illustrate c-MYC levels in ALCL cell lines after treatment with Formula 2 at various concentrations for eight (8) hours. X-axis, cell lines. Y-axis, mRNA quantities, relative to GAPDH.

FIG. 26 illustrates c-MYC levels in ALCL cell lines after treatment with Formula 2 at various concentrations for twenty four (24) hours. X-axis, cell lines. Y-axis, mRNA quantities, relative to GAPDH.

### DETAILED DESCRIPTION OF THE INVENTION

In one embodiment, the invention comprises a method of treating B-cell malignant cancers or T-cell malignant cancers by administering to a patient a pharmaceutically acceptable amount of a composition comprising a thienotriazolodiazepine compound, said thienotriazolodiazepine compound being (S)-2-[4-(4-chlorophenyl)-2,3,9-trimethyl-6H-thieno[3,2-f][1,2,4]triazolo -[4,3-a][1,4]diazepin-6-yl]-N-(4-hydroxyphenyl)acetamide (also known as Y-803 and OTX-015) represented by the following Formula (2):

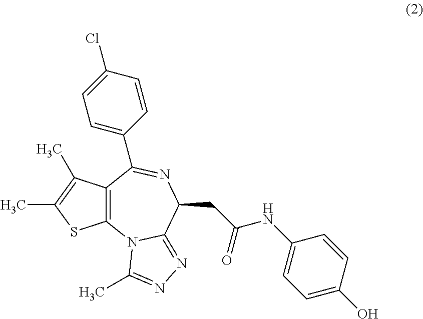 [SMILES: Cc1sc2c(c1C)C(c1ccc(Cl)cc1)=N[C@@H](CC(=O)Nc1ccc(O)cc1)c1nnc(C)n1-2]

The preparation of the compound represented by Formula 2 an be accomplished by chemical synthesis by those of ordinary skill in the art according to the methods previously described in the art, including those described in U.S. Pat. No. 5,712,274, which is incorporated by reference here in its entirety.

In another embodiment, the invention comprises a method of treating B-cell malignant cancers by administering to a patient a pharmaceutical composition where the active ingredient is represented by Formula 2 and where the patient is a human.

In another embodiment, the invention comprises a method of treating diffuse large B-cell lymphoma by administering to a patient a pharmaceutical composition where the active ingredient is represented by Formula 2. In one such embodiment, the patient is a human.

In another embodiment, the invention comprises a method of treating splenic marginal zone lymphoma by administering to a patient a pharmaceutical composition where the active ingredient is represented by Formula 1. In one such embodiment, the patient is a human.

In another embodiment, the invention comprises a method of treating anaplastic large T-cell lymphoma by administering to a patient a pharmaceutical composition where the active ingredient is represented by Formula 2. In one such embodiment, the patient is a human.

The invention is further described by the following non-limiting examples, which illustrate the unexpected results of the methods of treatment.

### EXAMPLES

### Example 1

The activity of Formula 2 was evaluated in four mantle cell lymphoma (MCL), ten diffuse large B-cell lymphoma (DLBCL) established human cell lines, a set of multiple myeloma (MM) cell lines, three splenic marginal zone lymphoma (SMZL) cell lines and eight anaplastic large T-cell lymphoma (ALCL) cell lines. Cells were exposed to increasing doses of the compound for 72 hours. Human cell lines derived from MCL, DLBCL, MM, SMZL and ALCL were cultured according to the conditions given in Table 1.

TABLE 1
Cell lines and growth medium

| Cell line | Histology | Growth Medium |
|---|---|---|
| DoHH2 | DLBCL | RPMI-1640 (GIBCO Invitrogen, Basel, |
| | | Switzerland) |
| Karpas 422 | DLBCL | RPMI-1640 (GIBCO Invitrogen, Basel, |
| | | Switzerland) |
| OCI-Ly7 | DLBCL | RPMI-1640 (GIBCO Invitrogen, Basel, |
| | | Switzerland) |
| SU-DHL-2 | DLBCL | IMDM (GIBCO Invitrogen, Basel, |
| | | Switzerland) |
| SU-DHL-4 | DLBCL | RPMI-1640 (GIBCO Invitrogen, Basel, |
| | | Switzerland) |
| SU-DHL-5 | DLBCL | RPMI-1640 (GIBCO Invitrogen, Basel, |
| | | Switzerland) |
| SU-DHL-6 | DLBCL | RPMI-1640 (GIBCO Invitrogen, Basel, |
| | | Switzerland) |
| SU-DHL-7 | DLBCL | RPMI-1640 (GIBCO Invitrogen, Basel, |
| | | Switzerland) |
| U-2932 | DLBCL | RPMI-1640 (GIBCO Invitrogen, Basel, |
| | | Switzerland) |
| Val | DLBCL | RPMI-1640 (GIBCO Invitrogen, Basel, |
| | | Switzerland) |
| Granta | MCL | DMEM (GIBCO Invitrogen, Basel, |
| 519 | | Switzerland) |
| JeKo-1 | MCL | RPMI-1640 (GIBCO Invitrogen, Basel, |
| | | Switzerland) |
| MAVER-1 | MCL | RPMI-1640 (GIBCO Invitrogen, Basel, |
| | | Switzerland) |
| Rec-1 | MCL | RPMI-1640 (GIBCO Invitrogen, Basel, |
| | | Switzerland) |
| MM1S | MM | IMDM (GIBCO Invitrogen, Basel, |
| | | Switzerland) |
| RPMI | MM | IMDM (GIBCO Invitrogen, Basel, |
| 8226 | | Switzerland) |
| U-266 | MM | IMDM (GIBCO Invitrogen, Basel, |
| | | Switzerland) |
| L82 | ALCL | |
| FE-PD | ALCL | |
| MAC1 | ALCL | |
| Karpas | ALCL | |
| 299 | | |
| SUPM2 | ALCL | |
| T5 | ALCL | |

All growth media were supplemented with fetal calf serum (10%) and penicillin-streptomycin-neomycin (˜5,000 units penicillin, 5 mg streptomycin and 10 mg neomycin/mL, Sigma) and L-glutamine (1%).

The proliferation assay was performed using the following procedure. Cells were seeded into 96-well plates at the density of 104 per well. Formula 2 (Oncoethix SA, Lausanne, Switzerland) was dissolved in DMSO as a stock solution of 10 mM and divided in aliquots stored at −80° C. For each experiment, an aliquot of the stock solution was thawed and used within 2 to 3 days. Formula 2 was serially diluted in tissue culture media, added to cells (in five replicates) and incubated for 72 hours at 37° C. Control cells were treated with equal amounts of DMSO. The 3-(4,5-dimethylthiazol-2-yl)-2,5-diphenyltetrazolium bromide (MTT) (Sigma, Buchs, Switzerland) was prepared as a stock of 5 mg/mL in phosphate-buffered saline (PBS) and filter-sterilized. An amount of MTT solution equal to 0.5 mg/mL was then added to each well and incubated in the dark at 37° C. for 4 hours. Cells were then lysed with 25% sodium dodecylsulfate (SDS) lysis buffer and absorbance was read at 570 nm on a Beckman Coulter-AD340 instrument. Three independent experiments were run for each cell line. The doses corresponding to the IC50 were estimated by fitting a sigmoidal model through the dose response curve using the R statistical package (R: A Language and Environment for Statistical Computing, R Foundation for Statistical Computing, Vienna, Austria).

Cell death was evaluated as follows. Cells were treated with DMSO or different doses of Formula 2 for 72 hours, harvested and washed once in PBS and then stained with propidium iodide (PI 1 μg/ml, Sigma) in PBS and analyzed using a FACScan flow cytometer (Becton Dickinson, Mountain View, Calif., USA). The analysis of the percentage of cell death was performed using CellQuest Pro software (Becton Dickinson).

Cell cycle analysis was performed using the following procedure. Cells were treated with DMSO or different doses of Formula 2 for 24 hours, harvested and washed once in PBS and then fixed in 80% ethanol at 4° C. for at least one hour. Cells were stained with propidium iodide (PI 50 μg/ml, Sigma) in PBS containing RNAse-A (75 kU/ml, Sigma) and analyzed for DNA content using a FACScan flow cytometer (Becton Dickinson). Cell cycle analysis was performed using the ModFit LT software package (Verity Software House, Inc., Topsham, Me., USA).

Western blotting analysis was performed as follows. Cells were solubilized in hot SDS lysis buffer (2.5% SDS, Tris-Hcl pH 7.4) and sonicated for 15 seconds. The protein content in the different samples was determined using the bicinchoninic acid (BCA) protein assay (Pierce Chemical Co., Rockford, Ill., USA). Lysates (40 μg) were fractionated by SDS-PAGE using 8% polyacrylamide gels, based upon the expected molecular weight. The resolved proteins were blotted to a nitrocellulose membrane by electric transfer, and the membranes were blocked in TBS-T buffer consisting of 20 mM trisaminomethane-HCl [pH 7.6], 137 mM NaCl, 0.1% polyoxyethylene sorbitan monolaurate (0.1% Tween 20) and containing 5% bovine serum albumin (BSA) for one hour. Membranes were incubated with primary antibodies diluted in TBS-T overnight. The following antibodies were used: anti-BRD2 (ab37633, AbCam, Cambridge, UK), anti-BRD3 (ab56342, AbCam), anti-BRD4 (ab75898, AbCam) and anti-a-GAPDH (MAB374, Millipore, Billerica, Mass., USA). Membranes were washed in TBS-T three times for ten minutes each and then incubated in TBS-T containing the appropriate horseradish peroxidase-conjugated anti-mouse or anti-rabbit secondary antibodies (Amersham Life Science, Arlington Heights, USA) for one hour. The membranes were washed three times for ten minutes each in TBS-T and then processed for enhanced chemiluminescence detection according to the manufacturer's instructions (Amersham Life Science). Equal loading of samples was confirmed by probing for GAPDH.

RNA was extracted using the RNA easy kit (Qiagen AG, Hombrechtikon, Switzerland). The concentration of total RNA was determined spectrophotometrically at 260 nm using a NanoDrop spectrometer (NanoDrop Technologies, Wilmington, Del., USA). One microgram of total RNA was reverse-transcribed using the Superscript First-Strand Synthesis System for real-time PCR kit (Invitrogen, Karlsruhe, Germany) according to the manufacturer's instructions. PCR amplification was performed using Fast SYBR Green Master Mix on a StepOnePlus real-time PCR System (Applied Biosystems, Foster City, Calif., USA). Primer sets (Table 2) were designed using the Primer3 software package (Rozen, S., Skaletsky, H. Primer3 on the WWW for general users and for biologist programmers. In: Misener, S., Krawetz, S. A., Eds. Methods in Molecular Biology, Vol. 132: Bioinformatics Methods and Protocols. Totowa, N.J., USA: Humana Press Inc., 2000, pp. 365-386). All samples were analyzed in triplicate. The relative quantity of the specific mRNA for each sample was calculated based on mean cycle threshold (Ct) values using the delta-delta Ct with a correction for experimental variations by normalization to the housekeeping gene GAPDH.

TABLE 2
Sequences of Used Primers

| | |
|---|---|
| BRD2-F | 5′-ACTTGGCCTGCATGACTACC-3′ |
| BRD2-R | 5′-CTGTAGCTTTCGTGCCATTG-3′ |
| BRD3-F | 5′-CAACCATCACTGCAAACGTC-3′ |
| BRD3-R | 5′-GGGAGTGGTTGTGTCTGCTT-3′ |
| BRD4-F | 5′-AGTCATCCAGCACCACCATT-3′ |
| BRD4-R | 5′-TCTTAGGCTGGACGTTTTGC-3′ |
| MYC-F | 5′-GGTGCTCCATGAGGAGACA-3′ |
| MYC-R | 5′-CCTGCCTCTTTTCCACAGAA-3′ |

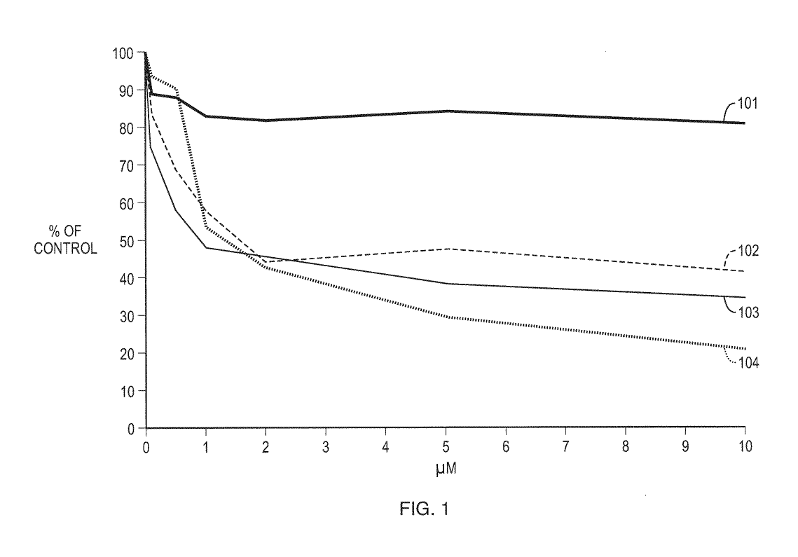

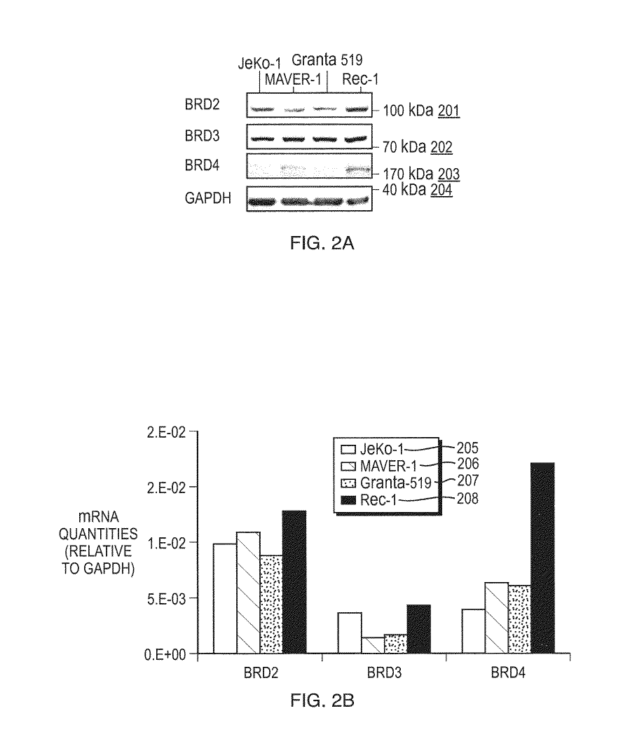

FIG. 1 shows the MTT results for the MCL cell lines. The corresponding estimated IC50 values are given for each MCL cell line that follows in parenthesis: Granta-519 (>15 μM), JeKo-1 (2.787 μM), MAVER-1 (1.224 μM), Rec-1 (1.224 μM). Formula 2 caused a dose-dependent decrease in cell viability in three MCL cell lines, with IC50 between 1.2-2.7 μM. The Granta-519 cell line did show any response when exposed to doses of up to 10 μM. All four cell lines expressed detectable levels of BRD2, BRD3, and BRD4, both at the RNA and the protein level (FIGS. 2A and 2B). Of interest, Rec-1, one of the cell lines with the lowest IC50, had the highest levels of all three BRDs, especially of BRD4 (FIG. 2B). These results indicate that these MCL cell lines are insensitive to Formula 2.

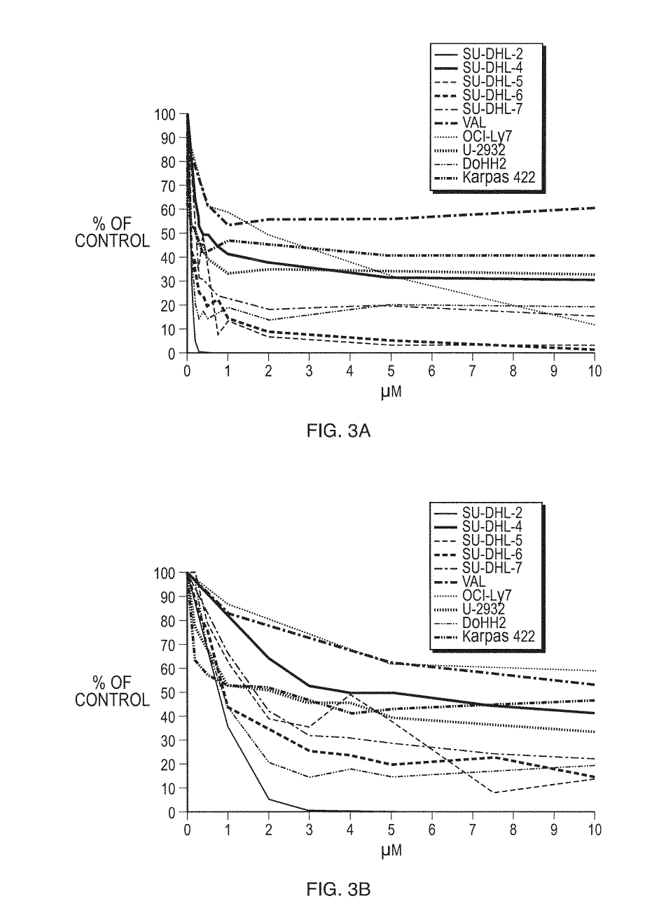

FIGS. 3A and 3B present the MTT data obtained in the DLBCL cell lines. The corresponding estimated IC50 values are given for each MCL cell line that follows in parenthesis: VAL (>12.68 μM), OCI-Ly7 (1.387 μM), SU-DHL-4 (0.607 μM), Karpass 422 (0.277 μM), U-2932 (0.255 μM), SU-DHL-5 (0.189 μM), SU-DHL-7 (0.132 μM), SU-DHL-6 (0.11 μM), DoHH2 (0.09 μM), SU-DHL-2 (0.069 μM). Formula 2 caused a dose-dependent decrease in cell viability in all the cell lines. Seven cell lines appeared very sensitive with IC50 values lower than 0.3 μM. Two cell lines had IC50 between 0.6 and 1.4. Only one cell line, VAL, despite showing a reduction in cell proliferation of over 40%, did not reach the IC50 when exposed to doses of up to 10 μM. Importantly, there were no differences in the sensitivity between cell lines derived from DLBCL of the germinal center type or of the activated B-cell like.

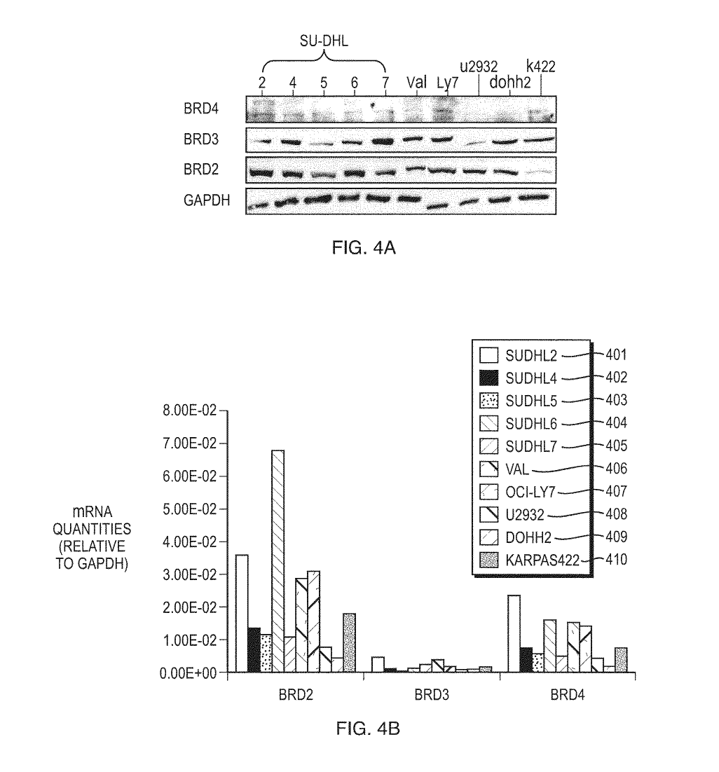

BRD2, BRD3, and BRD4 were expressed at variable levels in all the cell lines, at both RNA and protein level (FIGS. 4A and 4B). No clear correlation between response and expression levels could be observed. However, two of the most sensitive cell lines, SU-DHL-2 and SU-DHL-6, displayed high levels of BRD3/BRD4 and of BRD2, respectively (FIG. 4B).

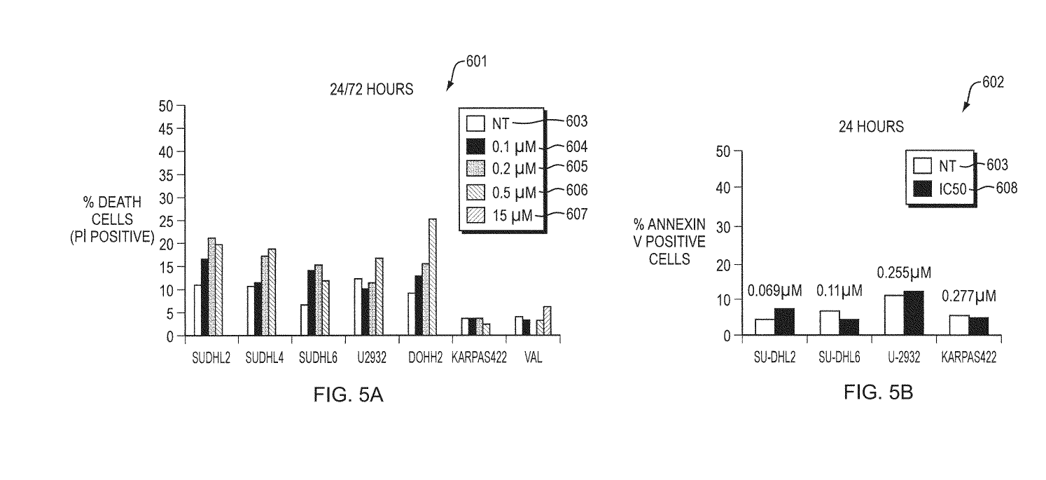

In order to investigate the possible effect of cytotoxic effect of Formula 2 on the DLBCL cell lines, the degree of cell death after exposure to the compound for 24 and 72 hours at doses in the range of 0.1-15 μM was evaluated, reflecting the observed IC50 values (FIGS. 5A and 5B). The data suggested that Formula 2 induces cell death only in a small percentage of the cell showing the lowest IC50 (SU-DHL-2 and DoHH2).

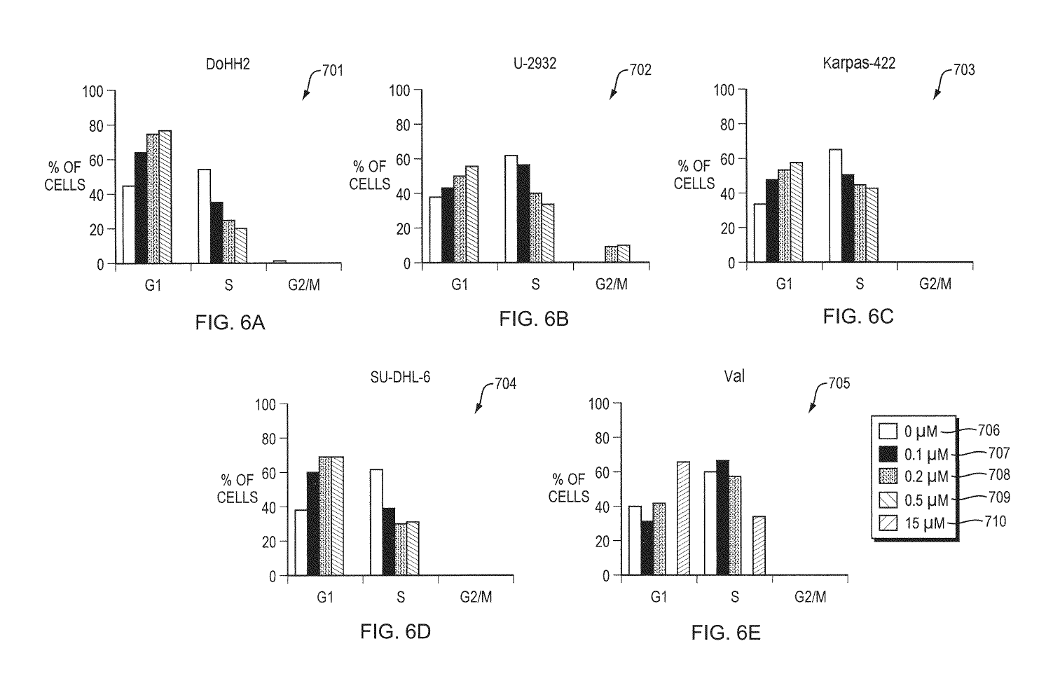

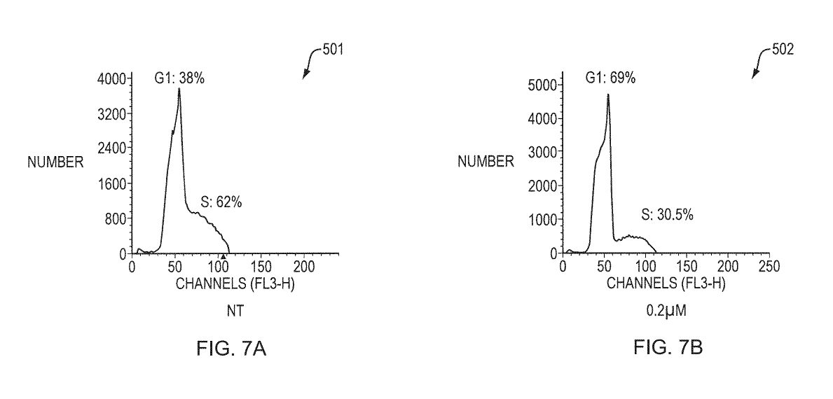

Not having observed the induction of massive cell death despite the important effect on cell viability, the effect of Formula 2 on the cell cycle was investigated (FIG. 6). Experiments performed on five DLBCL cell lines showed that Formula 2 induced a G1-arrest in a dose-dependent manner. FIG. 7 illustrates representative flow cytometry profiles of untreated control cells and SU-DHL-6 cells treated for 24 hours with 0.2 in μM of Formula 2. The data obtained so far suggest that Formula 2 has anti-tumor action on DLBCL cell lines and its activity might be mainly cytostatic.

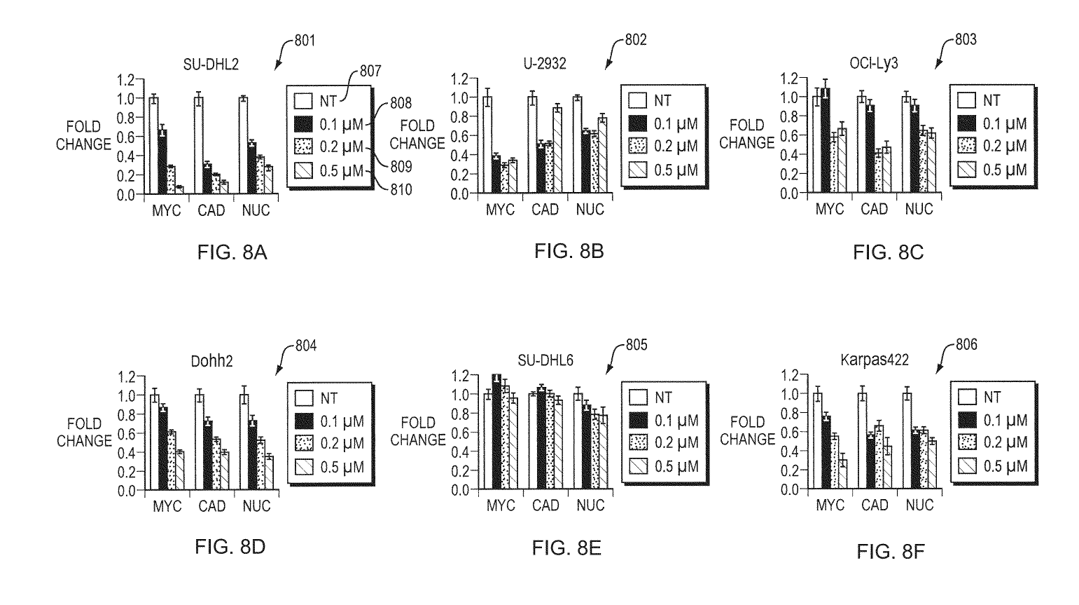

FIGS. 8A-8F illustrate reductions of c-MYC, CAD, and NUC mRNA levels after increasing doses of Formula 2 in six DLBCL cell lines, SU-DHL-2, U-2932, OCI-Ly3, DoHH₂, SU-DHL-6 and Karpas 422. c-MYC was down-regulated in five of the six cell lines after 24 hours of treatment.

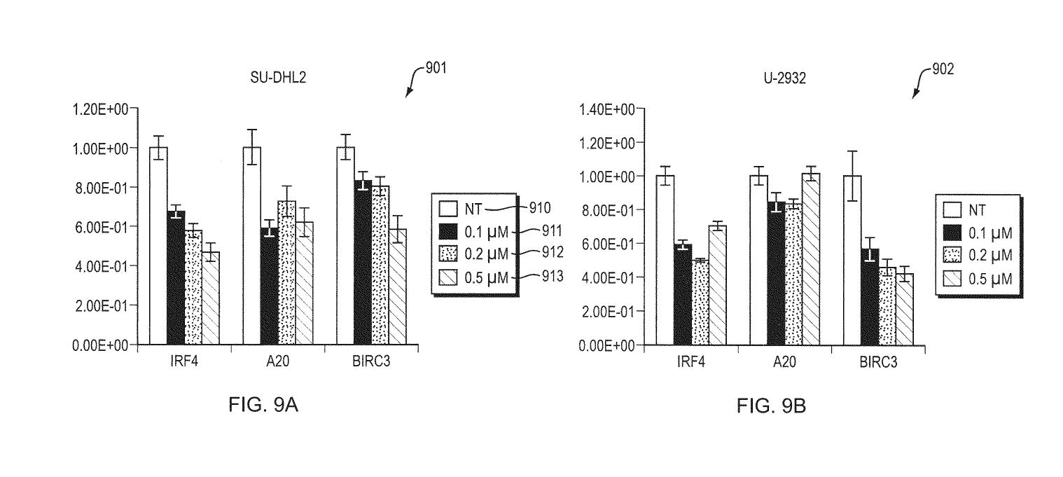

BRD4 co-activates transcriptional activation of NF-B via specific binding to acetylated ReIA. BRD4 KD suppresses NF-B related gene expression Huang B, Yang X D, Zhou M M, Ozato K, Chen L F: Brd4 coactivates transcriptional activation of NFκB via specific binding to acetylated Re1A. Mol Cell Biol 2009; 29:1375-1387. FIG. 9 illustrates the partial down-regulation of NFκB target genes after treatment with Formula 2 in two DLBCL cell lines.

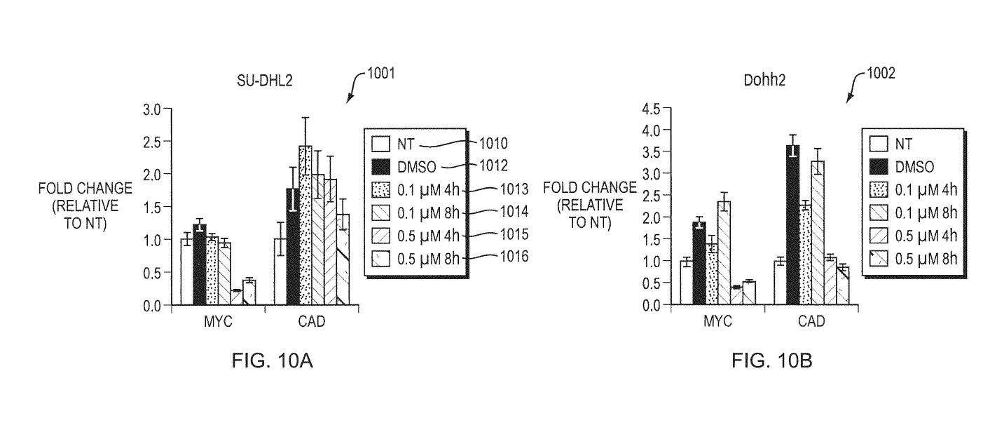

FIG. 10 illustrates gene expression profiles before and after exposure to various concentrations of Formula 2 in two sensitive models (SU-DHL2 and DoHH2).

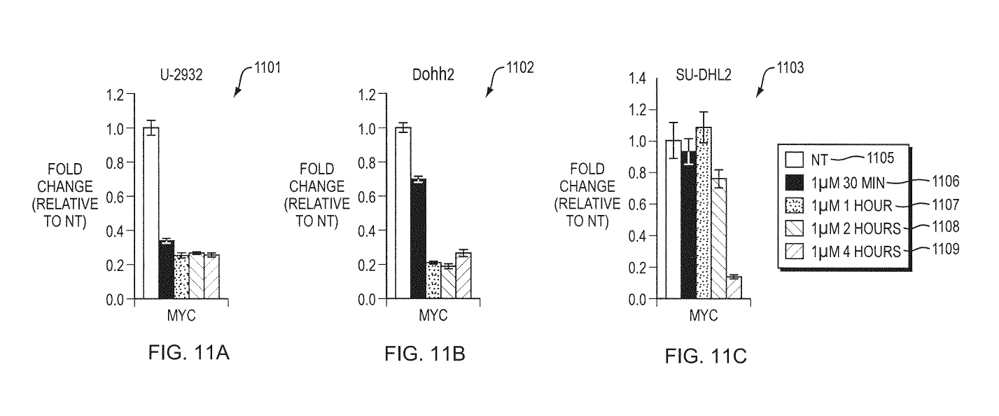

FIG. 11 illustrates the down-regulation of c-MYC in two of three DLBCL cell lines after 1 hour of treatment with Formula 2.

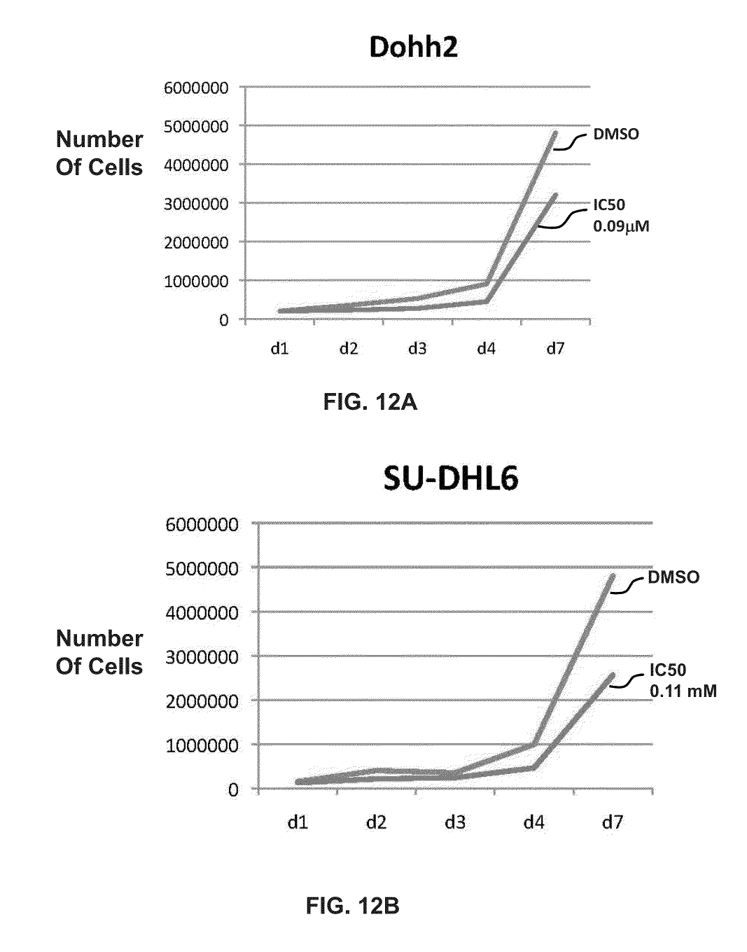

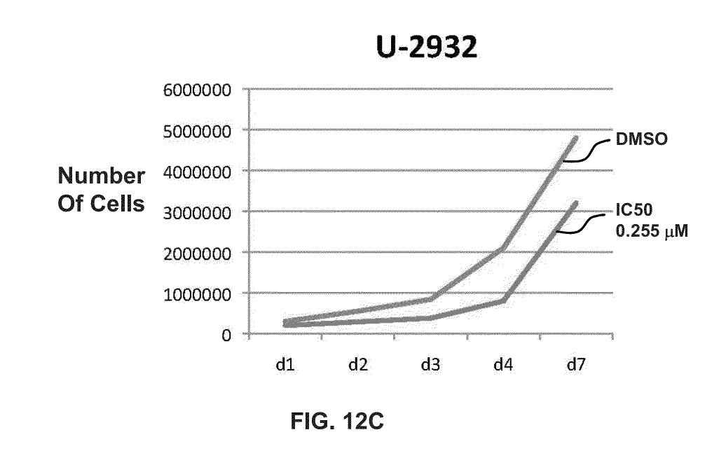

FIGS. 12A-12C illustrate the effect of Formula 2 on the proliferation of DLBCL cell lines, DoHH2, U-2932 and SU-DHL-6, with time after 24 hour treatment with IC50 dose of Formula 2 followed by wash-out.

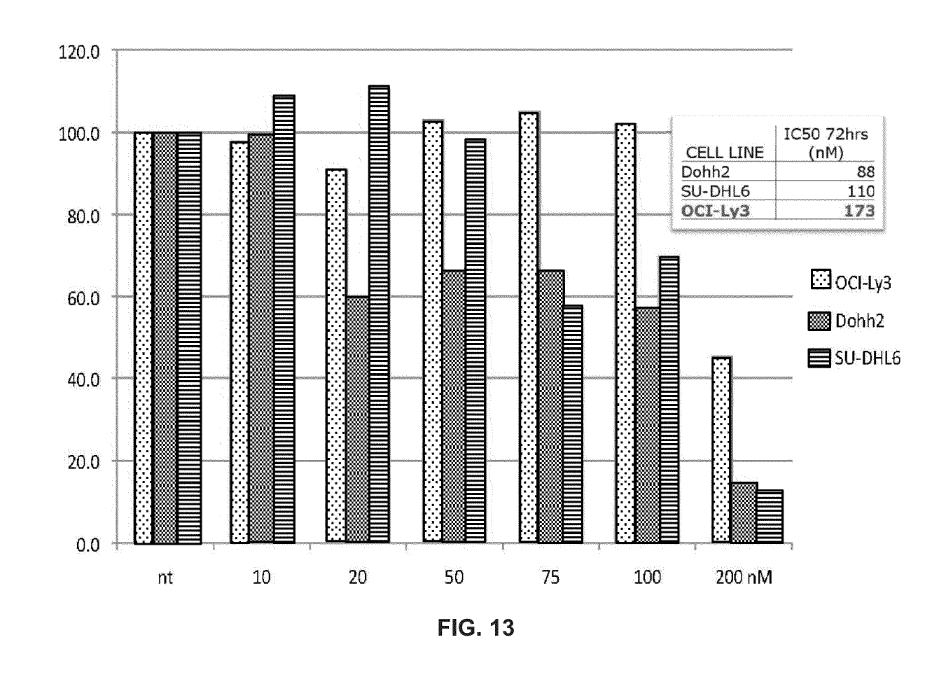

FIG. 13 illustrates the effect on three DLBCL cell lines after six (6) days of exposure of Formula 2.

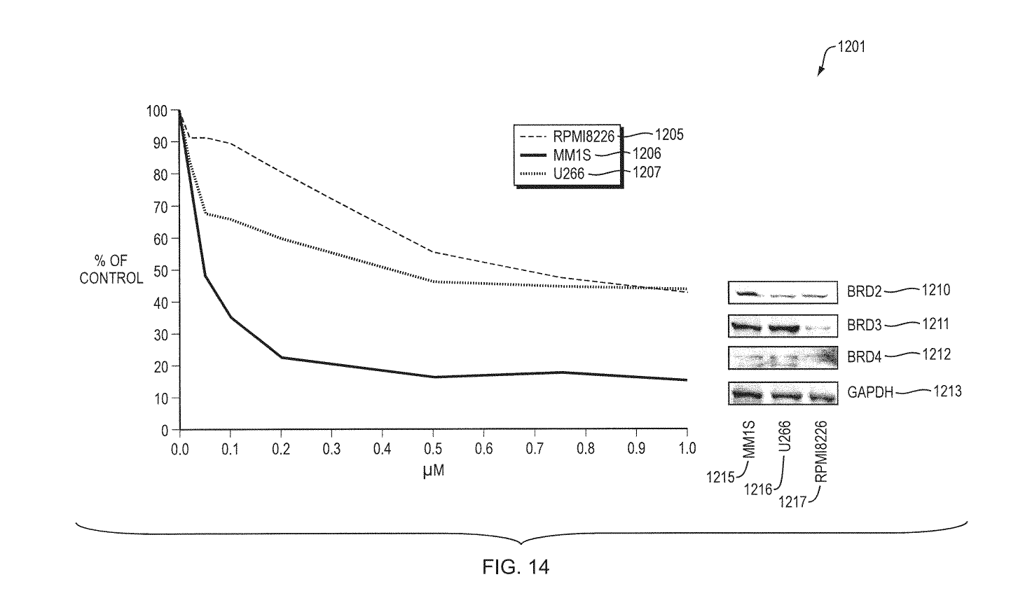

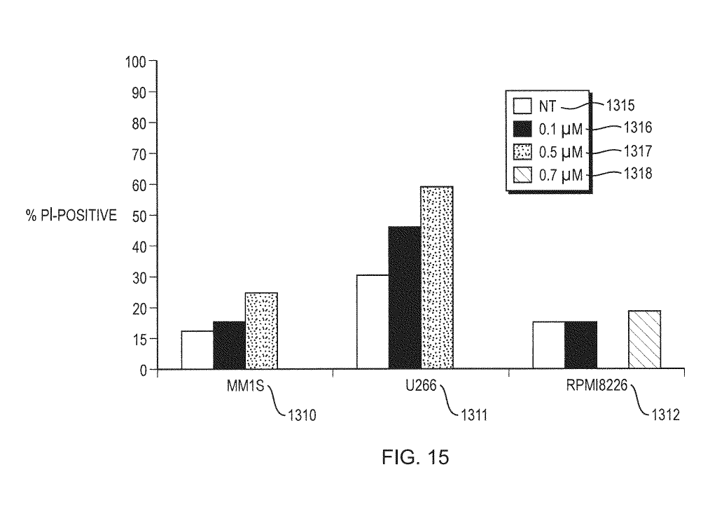

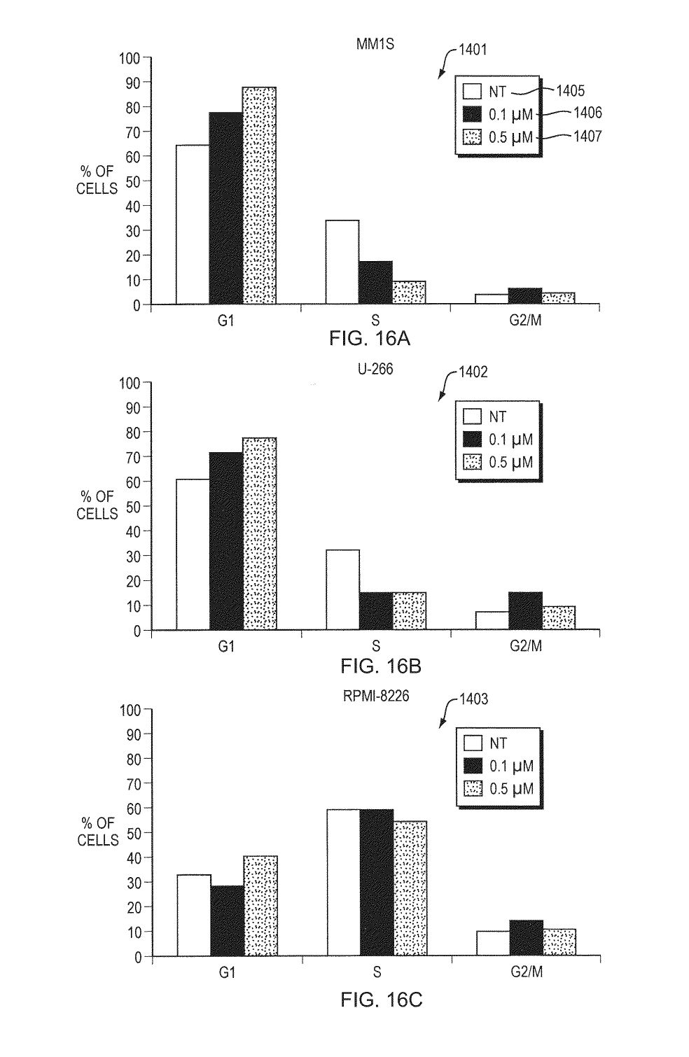

The activity of Formula 2 was also evaluated in MM cell lines. The MTT assay showed a reduction in cell viability in all the cell lines, with an IC50 between 0.06 and 0.7 μM (FIG. 15). The corresponding estimated IC50 values are given for each MM cell line that follows in parenthesis: RPMI8226 (0.7 μM), U266 (0.449 μM), MM1S (0.059 μM). All the BRD factors were expressed (FIG. 14). Similarly to what was observed in DLBCL, MM cell lines exposed to Formula 2 did not show an important increase of cell death (FIG. 15). Also, two of the three cell lines presented significant reduction of S-phase at cell cycle analysis (FIG. 16).

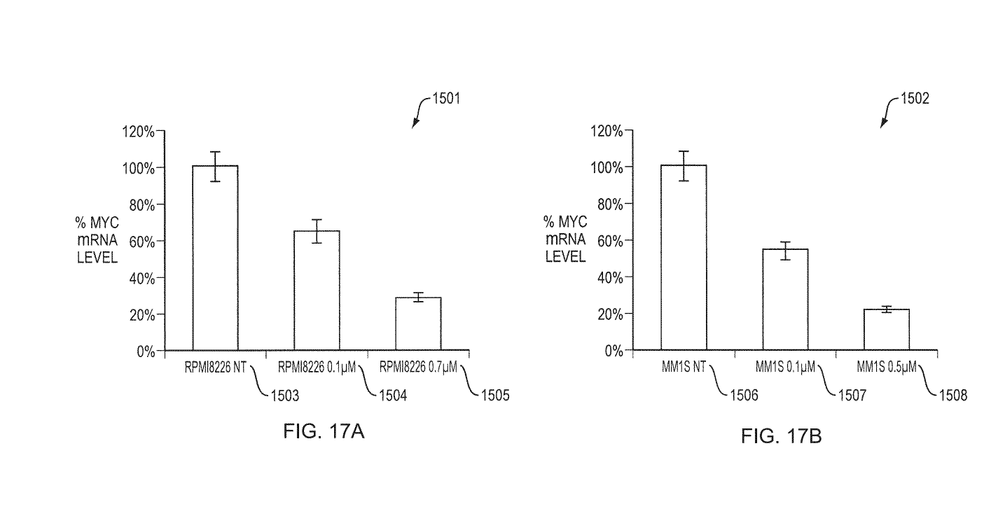

Considering the reported down-regulation of MYC following treatment of MM cell lines with the BRD inhibitor JQ1 2, MYC levels were evaluated after exposure to Formula 2 in RPM18226 and in MMS1 cell lines. Both cell lines presented a significant reduction of MYC mRNA levels in a dose-dependent manner at 24 hours (FIG. 17).

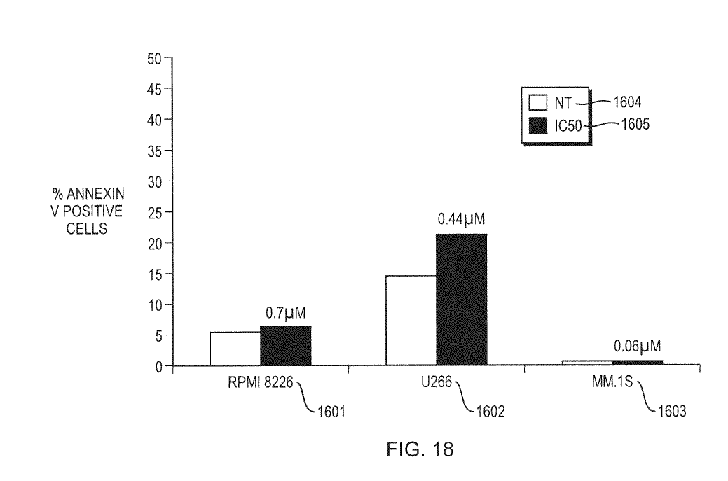

FIG. 18 illustrates the % Annexin V positive cells obtained after doses of Formula 2 at IC50 and 24 hours. The observed results suggest that Formula 2 exhibits a cytostatic effect on MM cell lines as opposed to a cytotoxic effect.

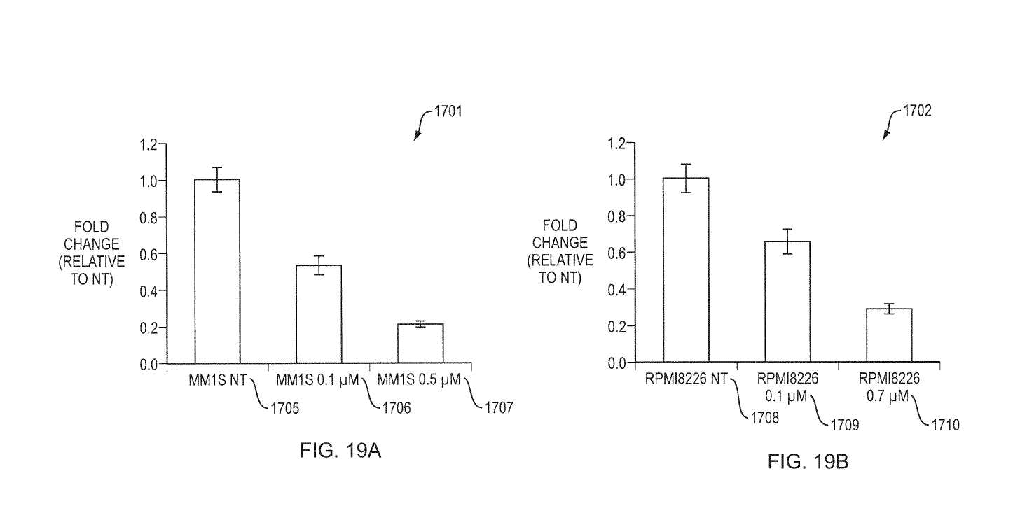

FIG. 19 illustrates c-MYC levels in MM cell lines. Formula 2 is observed to induce down-regulation of c-MYC in a dose-dependent manner.

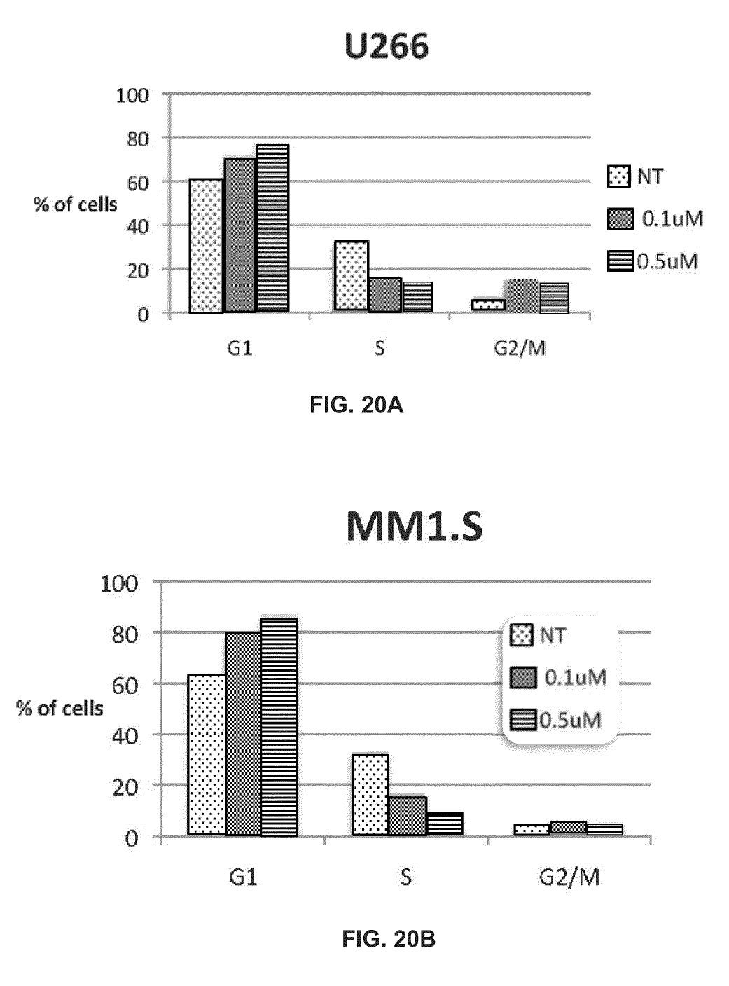

FIGS. 20A and 20B illustrate cell cycle alterations induced by Formula 2 in MM cell lines. Representative histograms of flow cytometry profiles of untreated control cells and cells treated for 24 h with different doses of Formula 2. X-axis, cell lines. Y-axis, percentage of cells in each cell cycle phase.

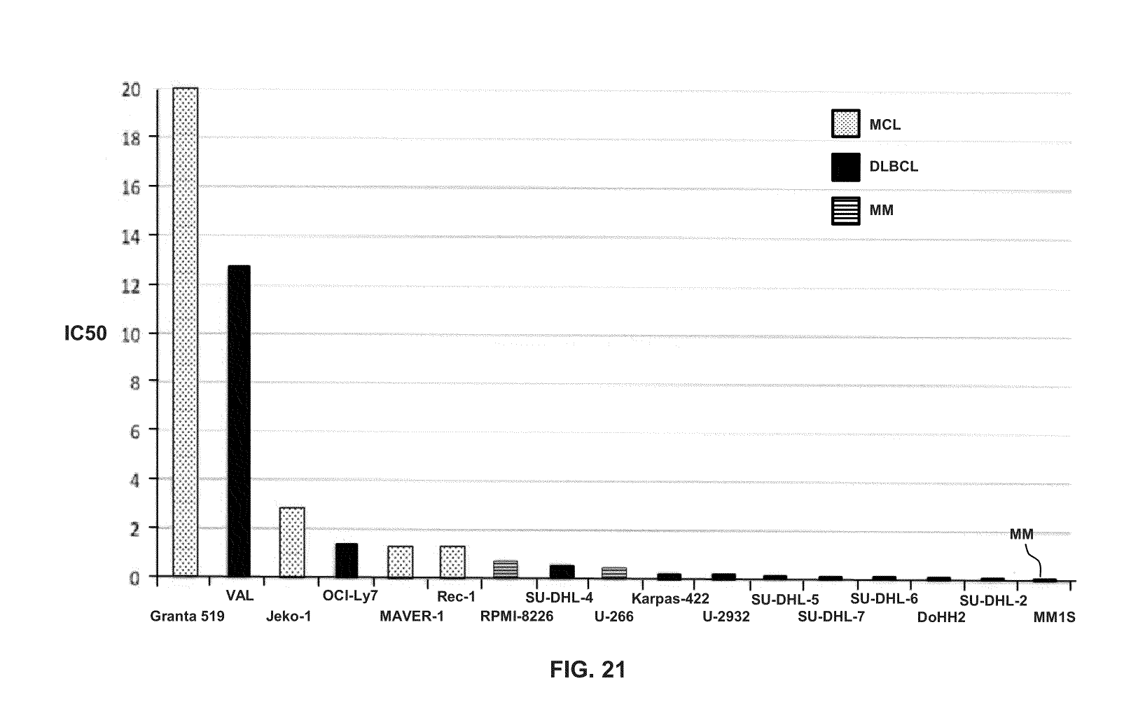

FIG. 21 illustrates a plot of all IC50 values for MCL, DLBCL and MM cell lines sorted by increasing sensitivity to Formula 2.

### Example 2

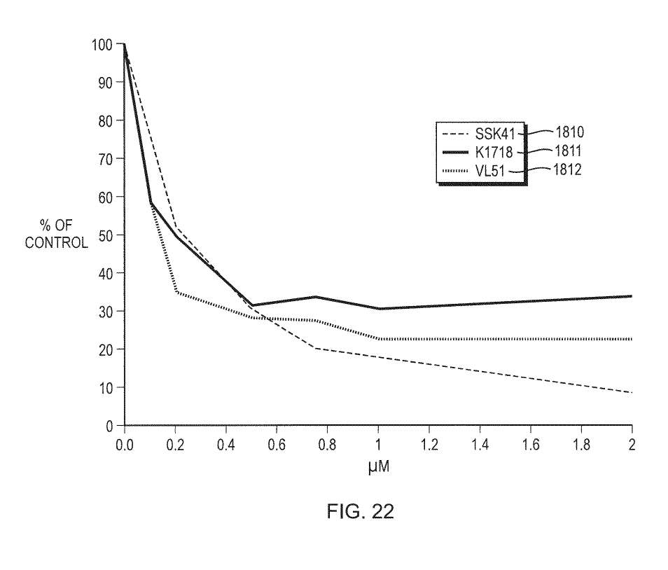

The activity of Formula 2 was also evaluated in SMZL cell lines using the procedures described in Example 1. FIG. 22 illustrates the effect of Formula 2 on the proliferation of SMZL cell lines. Formula 2 caused a dose-dependent decrease in cell viability in three SMZL cell lines

### Example 3

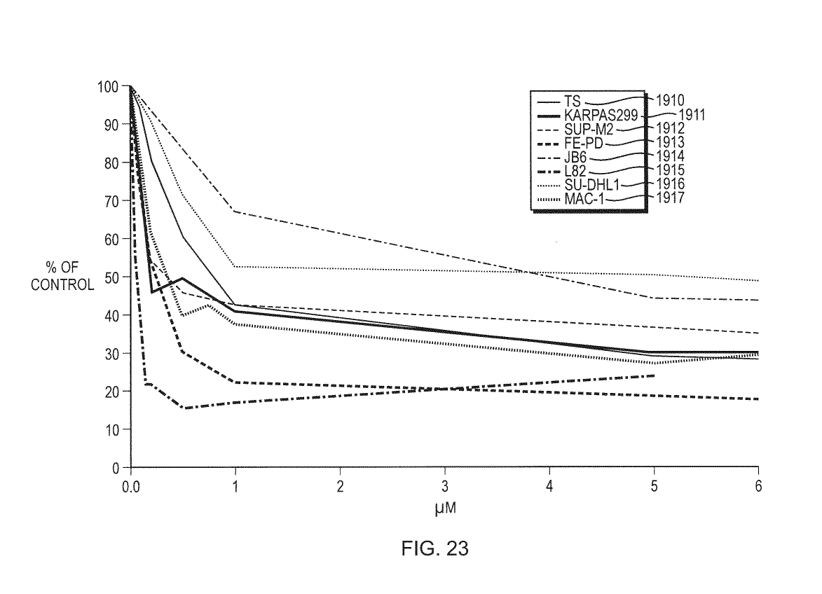

The activity of Formula 2 was also evaluated in ALCL cell lines using the procedures described in Example 1. FIG. 23 illustrates the effect of Formula 2 on the proliferation of ALCL cell lines. Formula 2 caused a dose-dependent decrease in cell viability in eight ALCL cell lines.

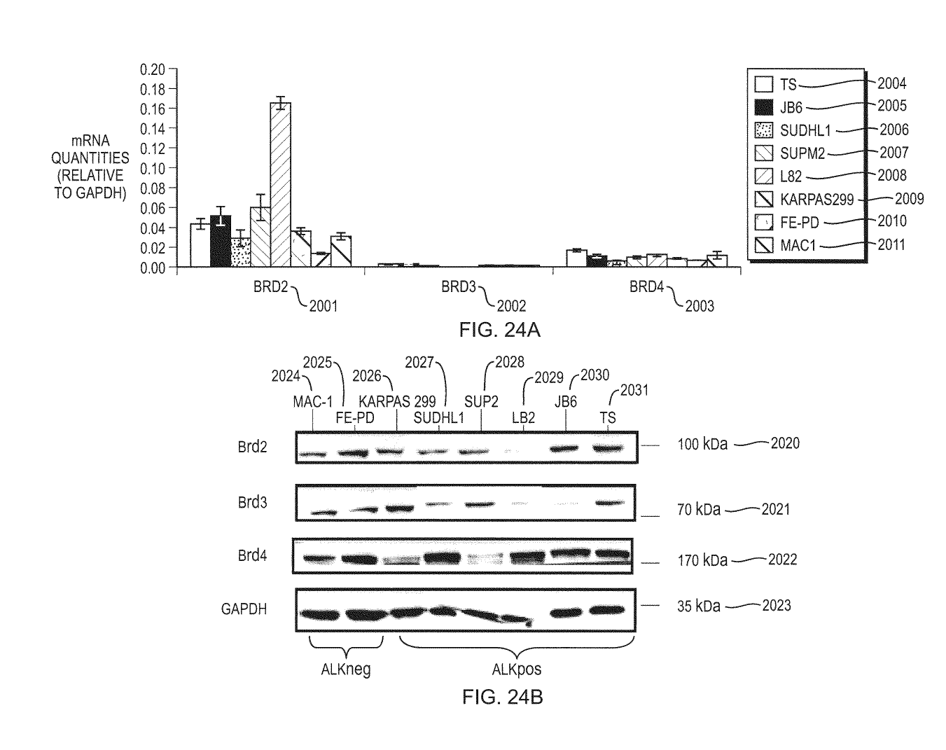

BRD2, BRD3, and BRD4 were expressed at variable levels in all the cell lines, at both RNA and protein level (FIGS. 24A and 24B). No clear correlation between response and expression levels could be observed.

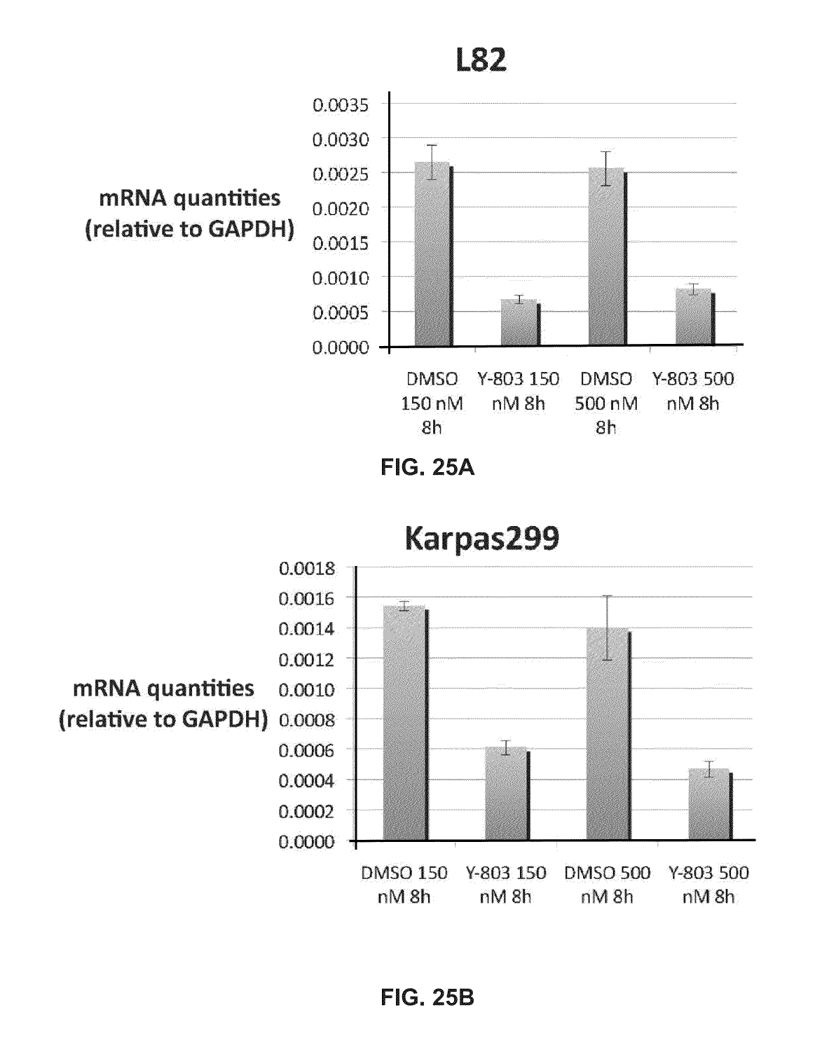

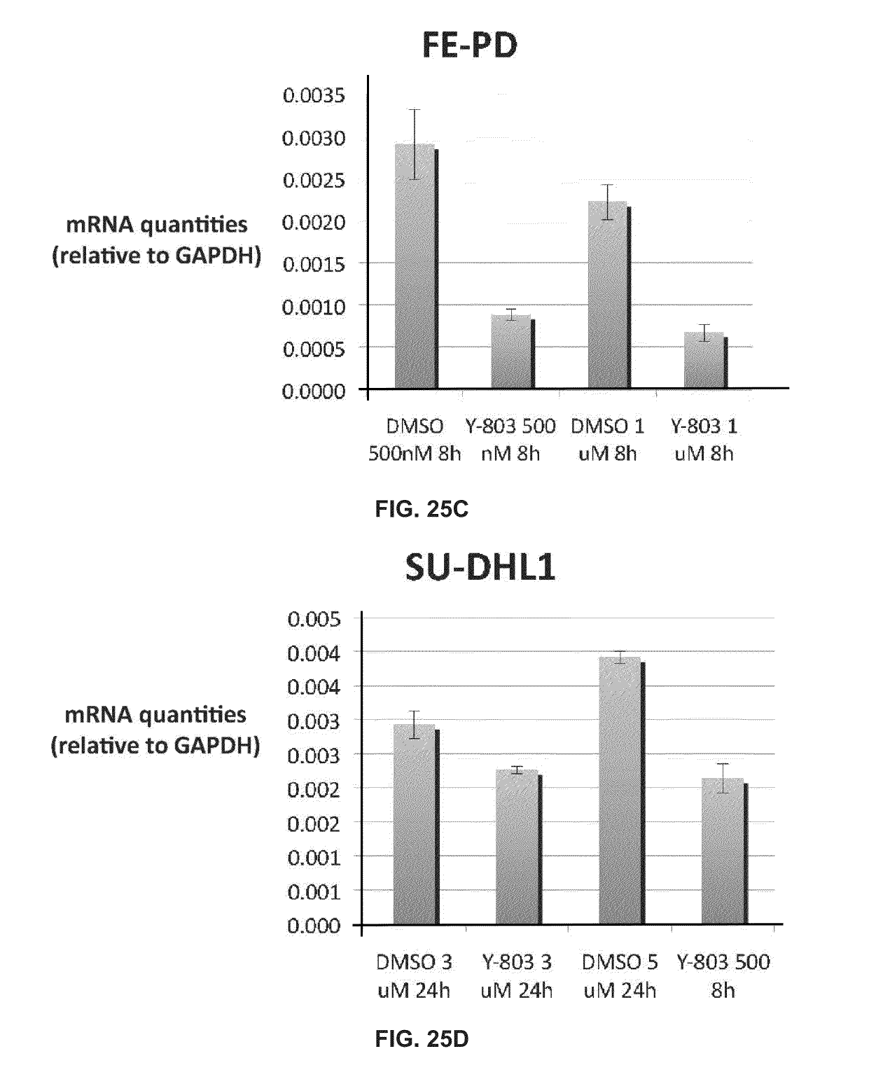

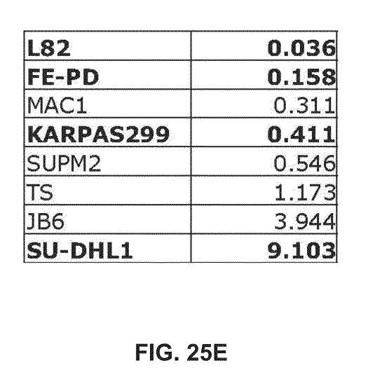

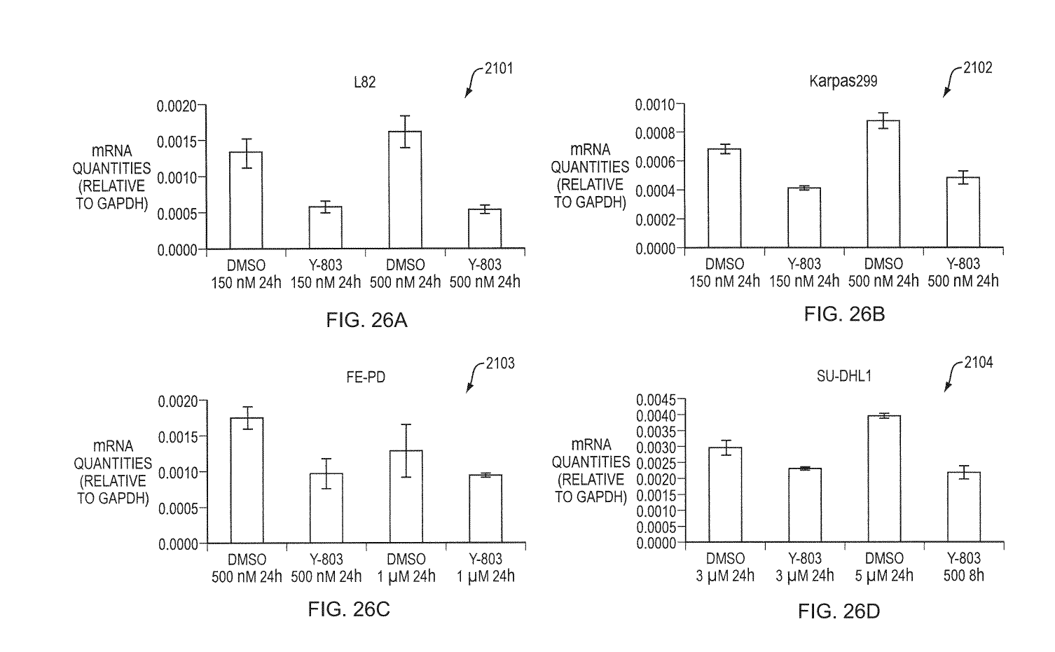

FIGS. 25A-25E illustrate c-MYC levels in ALCL cell lines after 8 hours of treatment with Formula 2. FIG. 26 illustrates c-MYC levels in ALCL cell lines after 24 hours of treatment with Formula 2. Formula 2 is observed to induce down-regulation of c-MYC in a dose-dependent manner.

The present disclosure may be embodied in other specific forms without departing from the spirit or essential attributes of the disclosure. Accordingly, reference should be made to the appended claims, rather than the foregoing specification, as indicating the scope of the disclosure. Although the foregoing description is directed to the preferred embodiments of the disclosure, it is noted that other variations and modification will be apparent to those skilled in the art, and may be made without departing from the spirit or scope of the disclosure.

What is claimed is:

1. A method of treating B-cell malignant cancer or T-cell malignant cancer comprising:
administering to a patient a pharmaceutically acceptable amount of a composition comprising a thienotriazolodiazepine compound as an active ingredient, said thienotriazolodiazepine compound being (S)-2-[4-(4-chlorophenyl)-2,3,9-trimethyl-6H-thieno[3,2-f][1,2,4]triazolo -[4,3-a][1,4]diazepin-6-yl]-N-(4-hydroxyphenyl)acetamide represented by the following Formula (2):

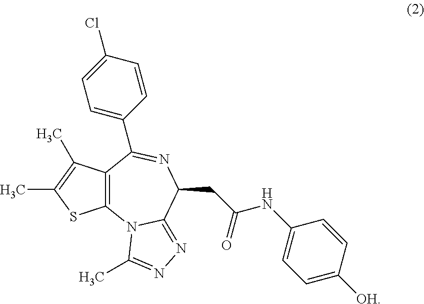 [SMILES: Cc1sc2c(c1C)C(c1ccc(Cl)cc1)=N[C@@H](CC(=O)Nc1ccc(O)cc1)c1nnc(C)n1-2]

2. The method of claim 1, wherein the patient is a human.

3. The method of claim 1, wherein the B-cell malignant cancer is diffuse large B-cell lymphoma.

4. The method of claim 1, wherein the B-cell malignant cancer is splenic marginal zone lymphoma.

5. The method of claim 1, wherein the T-cell malignant cancer is anaplastic large T-cell lymphoma.


SEQUENCE LISTING (8 sequences)

SEQ ID NO: 1 - DNA, 20 bp, Artificial Sequence
  Forward primer for BET bromodomain protein 2
acttggcctgcatgactacc

SEQ ID NO: 2 - DNA, 20 bp, Artificial Sequence
  Reverse primer for BET bromodomain protein 2
ctgtagctttcgtgccattg

SEQ ID NO: 3 - DNA, 20 bp, Artificial Sequence
  Forward primer for BET bromodomain protein 3
caaccatcactgcaaacgtc

SEQ ID NO: 4 - DNA, 20 bp, Artificial Sequence
  Reverse primer for BET bromodomain protein 3
gggagtggttgtgtctgctt

SEQ ID NO: 5 - DNA, 20 bp, Artificial Sequence
  Forward primer for BET bromodomain protein 4
agtcatccagcaccaccatt

SEQ ID NO: 6 - DNA, 20 bp, Artificial Sequence
  Reverse primer for BET bromodomain protein 4
tcttaggctggacgttttgc

SEQ ID NO: 7 - DNA, 19 bp, Artificial Sequence
  Forward primer for C-MYC oncogene
ggtgctccatgaggagaca

SEQ ID NO: 8 - DNA, 20 bp, Artificial Sequence
  Reverse primer for C-MYC oncogene
cctgcctcttttccacagaa